(19) United States
(12) Patent Application Publication
(10) Pub. No.: US 2003/0148963 A1
(43) Pub. Date: Aug. 7, 2003

(54) Use of etoposide

(76) Inventors: Andrej Tarkowski, Goteborg (SE); Margareta Verdrengh, Floda (SE)

Correspondence Address: Sammuels, Gauthier & Stevens LLP, Suite 3300, 225 Franklin Street, Boston, MA, 02110, US

(21) Appl. No.: 10/303,371

(22) Filed: Nov. 25, 2002

Related U.S. Application Data
(63) Continuation of application No. PCT/SE01/01217, filed on May 31, 2001.

(30) Foreign Application Priority Data
May 31, 2000 (SE) 0002045-3

(51) Int. Cl. A61K 31/7048

(52) U.S. Cl. 514/27

(57) ABSTRACT

The present invention relates to the use of etoposide at the manufacture of pharmaceutical compositions for the treatment of inflammations, such as arthritis in mammals, in particular humans.

### TECHNICAL FIELD

The present invention relates to a new use of etoposide in the treatment of inflammations, such as arthritis, in particular rheumatoid arthritis in mammals, including humans.

The object of the present invention is to obtain a possibility of alleviating and/or treating the symptoms of inflammations, such as arthritis, in particular rheumatoid arthritis in mammals, including humans.

Such symptoms are

1. Degenerative diseases having a significant inflammatory sign:

Alzheimer demens, Vascular demens, Stroke, Arteriosclerosis, Heart infarct, Heart insufficiency, Cardiomyopaties, Lumbago ischias

2. Inflammatoric diseases

Asthma bronchiale, Psoriasis, Chronic urticarial reactions, Primary lung alveolites

3. Chronic infection diseases having inflammatoric conditions (in which group of diseases etoposide should be used in combination with an adequate antibiotic agent or chemotherapeutic agent)

Tuberculosis, HIV, Infectious hepatitis, Tropical infection diseases e.g., leprae, schistosomiasis, Chagas disease

4. Autoimmune diseases

Rheumatoid arthritis, Systemic lupus erythematosus, Progressive systemic sclerosis Polymyositis/dermatomyositis/inclusions body myosotis, Ankylosing pelvospondylitis Primary vasculitis diseases, e.g., polyartheritis nodosa, Wegeners granulomatosis, Churg-Strauss vasculitis, Behcet syndrom, Polymyalgia rheumatica, Henoch-Schönlein purpure, microscopic vasculitis and others. Autoimmune hepatitis, e.g., chronic active hepatitis. Diabetes mellitus type I, Inflammatoric gastro intestinal diseases, Mb. Crohn and ulcerous colitis, Multiple sclerosis, Myastenia gravis, Hematological autoimmune diseases, e.g. idiopatic thrombocytopen purpure, Autoimmune hemolytic anaemia, autoimmune hemophilia. Nefrological autoimmune diseases, e.g., IgA nephritis, Primary uveites, iridocyclites

5. Prevention of/treatment at rejection of transplants

As will be shown in the following the etoposide can be used in suboptimal dosages of the drug. The dose of etoposide used for treating collagen arthritis this has by no means influenced the total amount of circulating monocytes. In spite of this it has been possible to eradicate completely the arthritis when the treatment was used at colagen II immunization and the number of arthritic mice has been halved when the treatment was begun during an already established clinical overt arthritis disease. These results means that considerably lower amounts/doses of etoposide (compared to oncological doses) have a pronounced positive effect at experimental inflammatory auto immune disease.

### PRIOR ART

Etoposide and its different derivatives is known as an anticancer agent in the form of a cytostatic agent causing cell death in cancer tumours and other cancer tissue. Thereby etoposide is administered in an amount of 50-100 mg/m² body area per day for a week. A normal body area is thereby 1.5 to 1.8 m² for an adult person, or, if expressed per kg bodyweight, 0.75-1.25 mg/kg bodyweight per day.

Study 1

Cells from the monocytes/macrophage lineage are together with granulocytes, the earliest type of leukocytes entering the tissue in response to invading pathogens. Once localized to a site of infection, macrophages phagocytize infectious organisms, act as antigen presenting cells, and secrete a large number of immuno regulatory products. It has previously been described a murine model of hematogenously induced Staphylococcus aureus induced sepsis and arthritis [6,9]. Mice given intravenously (i. v.) a single dose of the toxic shock syndrome toxin 1 (TSST-1)-secreting S. aureus LS-1 develop clinical signs of arthritis within 48 h Most of the deleterious effects of infection are not due to the direct cytotoxicity of bacteria on the host tissue but rather depend on the exaggerated host immune response. More specifically, T lymphocytes contribute to the development of the disease, since depletion of CD4⁺ T cells or T cell receptor-expressing cells improve the outcome of the disease [2,3,7]. Also B cells and B cell-derived cytokines contribute to the pathogenesis of S. aureus infection [261. It was recently demonstrated that the efficient recruitment of granulocytes in the early phase of the infection is critical for the beneficial outcome of the disease [24]. Another important cell population with phagocytic properties is the monocyte/macrophage. The majority of synovial cells in the cartilagesynovium junction participating in the destructive process, has the appearance of macrophages [6]. Using in situ hybridization techniques, it has been shown that the monocyte/macrophage-derived cytokines including mRNA for TNF- and for IL-1 are rapidly induced in the infected synovia [28]. Indeed, TNF is an important cytokine mediating septic arthritis [15].

The aim of the present study was to evaluate the role of macrophages in the host defence to invading bacteria. The results indicate a dual role of this cell population: on one hand absence of macrophages provides a more favourable outcome concerning arthritic lesions but on the other the clearance of bacteria by monocytes/n-macrophages is decreased resulting in increased mortality.

### MATERIALS AND METHODS

Mice

NMRI mice of both sexes, 6-8 weeks old, were obtained from B&K Universal AB (Stockholm, Sweden). They were housed in the animal facility of the Department of Rheumatology, University of Göteborg, under standard conditions of light and temperature and fed standard laboratory chow and water ad libitum.

Induction of Monocytopenia

Etoposide (Bristol Myers Squibb AB, Bromma, Sweden) s a drug which leads to a selective decrease of peripheral blood monocytes in rabbits and in mice [21,23]. Etoposide functions by inhibition of DNA topoisomerase II function, interrupting the late S/G2 phase of the cell cycle [20].

Etoposide was diluted 1:10 in PBS (0.13 M NaCl, 10 mM sodium phosphate (pH 7.4) from a stock solution of 20 mg/ml. A volume of 150-200 μl corresponding to 12.5 mg/kg was injected subcutaneously once a day during the course of the experiment, starting 3 days before i. v. inoculation with S. aureus. The dose of etoposide was chosen according to earlier studies [10]. The controls received PBS.

Hematological Analyses Mice were bled from the tail into heparinized tubes on days −3, −1, 3, and 6-7 after bacterial inoculation. The leukocytes were counted in a hemacytometer (Toa Medical Electronics, Kobe, Japan). Fifty microliters of the heparinized blood was analysed in a FACScan cytometer (Becton Dickinson, San Jose, Calif.) to determine the percentages of lymphocytes, granulocytes, and monocytes, respectively. The absolute numbers of different leukocyte subsets were then calculated from the total leukocyte counts.

Bacteria and Infection

S. aureus LS-1 strain was originally isolated from a swollen joint of a spontaneously arthritic NZB/W mouse [8]. The bacteria were cultured for 0.24 h on blood agar plates, replated for another 24 h, washed and kept frozen at −20° C. in PBS containing 5% bovine serum albumin (1BSA) and 10% dimethyl sulfoxide (C₂H60S) until used. Before use, the bacterial solution was thawed and washed in PBS. Viable count was done to check the number of bacteria in each bacterial solution mice were given i.v. injections (in the tail vein) of 0.2 ml of bacterial solution

Clinical Evaluation of Mortality and Arthritis

All the mice were monitored individually. Their limbs were inspected visually every day during the experiment. Arthritis was defined as visible joint erytbema and/or swelling of at least one joint. To evaluate the intensity of arthritis, a clinical scoring system of 0 to 3 points for each limb was used (1 point, mild swelling and/or erytema; 2 points, moderate swelling and erythema; 3 points, marked swelling and erytema). The arthritic index was constructed by adding the scores from all four limbs for each animal [1]. Previous study has shown that there is a good correlation between the clinical and histopathological appearance of arthritis [6]. The overall condition of each mouse was evaluated by assessing its weight, general appearance, alertness, and skin abnormalities.

Histopathological and Immunohistochemical Examinations

The mice were sacrificed 6 days after bacterial inoculation One front and one hind paw from each animal in both groups (etoposide-treated and controls) were removed. Histopathological examination was performed after routine fixation, decalcification, paraffin embedding, and staining with hematoxylin and eosin. All the slides were coded and the joints were studied with regard to synovial hypertrophy (defined as a synovial membrane thickness of more than two cell layers), pannus formation (synovial tissue overlaying the joint cartilage), and cartilage and bone destruction [1].

In another experiment, mice were killed by cervical dislocation 6 days after bacterial inoculation, all four limbs were removed and demineralized by a procedure detailed in an earlier report [16]. The demineralized specimens were mounted on cryostat chucks, frozen in isopentane prechilled by liquid nitrogen, and kept at −70° C. until cryosectioned. Six μm-thick sections were cut frontally to permit simultaneous inspection of most joints within the paw. All sections were fixed in cold acetone for 5 min, washed in PBS, and depleted of endogenous peroxidase by treatment with 0.3% H₂O₂ for 5 min. The sections were incubated overnight in a humid atmosphere at +4° C. with 50 μl portions of unlabelled rat monoclonal antibody specific for macrophages (Mac-3, clone M3/84 from PharMingen), diluted in PBS containing 1% BSA. Biotin-labelled rabbit antl-rat Ig diluted in PBS-BSA was used as secondary antibody. Binding of secondary antibodies was detected by stepwise incubation with avidin-biotin-peroxidase complexes (ABC) and a buffert containing 3-amino-9-ethyl-carbazole and H₂O₂. All sections were counterstained with Mayer's hematoxylin.

Determination of Bacterial Growth

On days 3 and 6-7 after S. aureus inoculation blood samples were obtained and the kidneys were aseptically removed. Appropriate dilutions were made and 0.1 ml of tissue suspensions and of whole blood was plated on agar plates containing 5% horse blood. Six days after the inoculation bacterial samples from the talocrural and radiocarpal joints were obtained and plated on agar plates, containing 7.5% sodium chloride. After incubation for 48 h at 37° C. the bacterial colonies were counted and tested for catalase and coagulase activity.

In Vitro and Ex Vivo Stimulation of Spleen Mononuclear Cells for Proliferation and Cytokine Production

Spleens were obtained from healthy NMRI mice. The preparation of spleen mononuclear cells was performed as previously described [27]. The cells (2×10⁶¹/Ml) were cultured in Iscove's complete medium (10% FCS, 5×10⁻⁵ M 2-mercaptoethanol, 2 mM L-glutamine, and 50 μg/ml gentamicin). The cultures were treated with etoposide in the concentrations 0, 1, 10, and 100 ELM and maintained in 24-well plates (Nunc, Roskiide, Denmark) at 37° C. in 5% CO₂ and 95% humidity during 30 min prior to stimulation with 1.25 μg/ml of Con A (ICN Biochemicals, Cleveland, Ohio) or with 10 μg/ml of highly purified TSST-1 (Toxin Technology, Sarasota, Fla.). The supernatants were collected after 24 h of incubation for analysis of IL-6 and TNF_{.}-. To determine the proliferative response, the cells were cultured in 96-well plates (Nunc) for 3 days. The cultures were pulsed with 1 μCi of (³H)TdR (Amersham, Bucks, UK) 12 h before harvest and calculation of TdR uptake in a beta counter.

To analyse the numbers of live cells at 24 h, the cell suspensions were stained with propidium iodide (5 μl of 100 μl/ml per 1×10⁶/ml of cell suspension) prior to analysis in a FACScan cytometer (Becton Dickinson).

In another experiment spleens were taken from mice treated with etoposide once a day for 3 days and non-treated controls. The spleen cells were cultured with Con A and TSST-1 as described above, and the supernatants analysed for IL-6 and TNM-production (24 h) as well as for proliferative responses (72 h).

Assessment of Cytokine Levels in Sera and Supernatants

IL-6 assay.

The cell line B 13,29, subclone B9, which is dependent on IL-6 for its growth, was used for IL-6 determinations [6]. The cells were seeded into microtiter plates at a concentration of 5000 cells/well and samples in different dilutions were added. One μCi of (3H) TdR (Amersham) was added after 68 h of culture, and the cells were harvested 4 h later. The results were compared with recombinant IL-6 standard (Genzyme, Cambridge, Mass.). The B9 cells were previously shown not to react with several recombinant cytolines including IL_{.}-1, IL_{.}-, L-2, IL-3, IL-5, granulocyte/macrophage CS17, TNF_{.}-, and IFN_{.}-, There was only a weak reactivity with IL-4 [14].

TNF_{.}- Assay.

The levels of TNF_{.}- were determined using sandwich enzyme-linked immunosorbent assays (ELISA) kit from R&D Systems Minneapolis, Minn.).

In Vivo Cell Mediated Inflammatory Responses

A) Immunization procedure and registration of delayed type hypersensitivity (TI). To assess the effect of monocytopenia on T cell mediated in vivo responses, the mice were sensitized by epicutaneous application of 50 μl of a mixture of absolute ethanol and acetone (3:1) containing 3% 4-ethoxyymethylene-2-phenyloxazolone (Sigma Chemical Co, St Louis, Mo.) on the shaved abdomen and thorax skin. Seven days after sensitization all the mice were challenged by application of 15 μl 1% oxazolone dissolved in olive oil on both sides of the right ear. Two days, the day before and 1 h before challenge the mice were pretreated with 12.5 mg/kg of etoposide. The thickness of the ear was measured before and 24 hours after challenge with an Oditest spring caliper (Kröplin, Schluchtern, Germany), as previously described [11]. The intensity of the DTH reaction was expressed (ear-thickness^{24 h}−ear thickness^{0 h})×10⁻³ cm.

B) Induction and Registration of Olive Oil Inflammation.

Olive oil induces in vivo a strong granulocyte mediated but T cell independent inflammatory response [18,24]. Inflammation was induced by injection of 30 μl olive oil (Apoteksbolaget AB, Göteborg, Sweden) intradermally in hind foot-pad. The mice were pretreated with etoposide every day starting 2 days before injection of olive oil. Foot-pad swelling was registered 24 h after injection using an Oditest spring caliper. The intensity of the olive oil induced inflammation was expressed (foot-pad thickness^{24 h}−foot-pad thickness^{0 h})×10⁻³ cm.

Statistics

Statistical comparisons were made by the Mann-Whitney U test and chi-square test with Yates correction. All values are reported as the means standard error of the mean (SEM).

Results

Effect of Monocytopenia on S. aureus-Induced Arthritis and Mortality

To analyse the impact of monocyte depletion on the course of systemic S. aureus infection, four experiments were performed using i. v. inoculation of 1-2×10⁷ CFU of S. aureus per mouse after pretreatment with etoposide for 3 days. The results are pooled since the outcome of each experiment turned out to be similar. Throughout the course of each experiment the etoposide-treated animals exhibited clinically a significantly less severe arthritis (FIG. 1A). Thus, already 2 days after bacterial inoculation the severity of arthritis was less pronounced in the etoposide-treated animals than in the controls. Also the frequency of arthritis was less pronounced in the etoposide-treated group during the course of the experiments. Six days after inoculation 70% of the controls exhibited clinical signs of arthritis versus 35% of the etoposide-treated animals. The histopathological examination of the joints obtained from S. aureus inoculated mice confirmed the clinical findings. In the monocytopenic group 4 out of 8 mice exhibited synovial hypertrophy versus 9 out of 10 in the control group. Cartilage and bone destructions were evident in 4 out of 8 etoposide-treated mice but in 8 out of 10 control animals. Immunohistochemical evaluation of the Joints from mice infected with S. aureus and treated with etoposide or PBS did not show any significant differences with respect to the numbers of Mac-3 positive cells (i.e. tissue bound macrophages) (data not shown). The monocytopenic mice developed a higher mortality than the controls, starting four days after bacterial inoculation (FIG. 1B). Even when a suboptimal dose of bacteria (1×10⁶ CFU) was used, the etoposide-treated group displayed a higher mortality rate than the controls. Thus, 8 days after bacterial inoculation 1 mouse out of 10 in the control group had died versus 6 out of 10 in the monocytopenic group. The weight loss was also more prominent in this group (data not shown).

Evaluation of Bacterial Growth

The bacterial elimination from the blood and kidneys was examined on days 3 and 6-7 after bacterial inoculation. The etoposide-treated mice displayed, compared to the control animals, an increased bacterial growth in both kidneys and blood at both occasions (Table 1). The joints were examined concerning growth of bacteria 6 days after inoculation. In the etoposide-treated group bacteria were recovered from 3 out of 8 mice versus 3 out of 10 in the control group.

The Impact of Etoposide on In Vitro and Ex Vivo Proliferative Response of Lymphocytes

To assess the impact of etoposide on the reactivity of immunocompetent cells in the absence of bacteria, spleen cells from naive mice were incubated with different concentrations of the drug. The proliferative responses to Con A and TSST-1, both compounds acting on T lymphocytes, significantly decreased upon increasing concentrations of etoposide (FIG. 2). This outcome reflected increased percentage of dead cells in wells with high concentrations of etoposide. In addition, spleen cells from non-infected, etoposide-treated mice displayed a lower proliferative response compared to the controls when stimulated with TSST-1 but not when stimulated with Con A (FIG. 3).

Etoposide Down-Regulates Production of Pro-Inflammatory Cytokines

Serum levels of IL-6 and TNF_{.}- were significantly lower in the etoposide-treated, infected animals than in the controls (Table 2). Since IL-6 and TNF_{.}- are secreted mainly by mononuclear phagocytes it was expected that production of IL-6 and TNF_{.}- in vitro, by mitogen and superantigen activated spleen mononuclear cells was significantly and dose-dependently suppressed by addition of etoposide (Table 3). In addition, in ex vivo spleen cell cultures recovered from etoposide-treated, non-infected mice the production of IL-6 and TIN-F_{.}- was significantly suppressed (Table 4).

Impact of Etoposide on Peripheral Blood-Eranulocyte-, Lymphocyte- and Monocyte Counts During—the Course of S. aureus Infection

As expected, treatment with etoposide significantly decreased the levels of circulating monocytes. During the course of infection the etoposide-treated animals remained monocytopenic while the levels of circulating monocytes did not change in the control mice (FIG. 4). The numbers of lymphocytes decreased throughout the experiment in both groups; however, more profoundly in the etoposide-treated group [5.6±0.4]×10⁶/ml on the day prior to bacterial inoculation versus [0.8±0.2]×10⁶/ml) seven days after infection. Corresponding figures for the controls were ([7.8±0.6]×10⁶/ml versus [2.4±0.3]×10⁶/ml). The controls exhibited an increase in the numbers of granulocytes during the course of infection ([1.6±0.3×10⁶/ml one day prior to injection with bacteria compared to [7.8±1.3]×10⁶/ml seven days after inoculation). The numbers of granulocytes in the etoposide-treated animals increased slightly throughout the course of the experiment [1.3±0.2]×10⁶/ml on day −1 compared to [1.6±0.4]×10⁶/Ml seven days after the inoculation).

In Vivo Cell-Mediated Inflammatory Responses

Since the outcome, of S. aureus induced infection and arthritis is both granulocyte and T cell dependent the in vivo functional effect of etoposide treatment was examined. The inflammatory swelling to intradermal injection with olive oil is a granulocyte-dependent inflammatory response. There were no differences in the swelling of the footpads in the non-infected monocytopenic mice (n=6) compared to the controls (n=6) ([128±131×10⁻³ versus [108±10]×10⁻³ cm). The DIH reaction to oxazolone, a T cell mediated reaction, was also similar in both study groups ([32±6]×10⁻³ versus [33±61×10⁻³ cm).

Discussion

In the present study the role of cells originating from monocyte/macrophage lineage in the pathogenesis of S. aureus infection was evaluated. Activated macrophage kill microorganisms by plagocytosis and generation of reactive oxygen species, become more efficient antigen presenting cells, produce mediators such as cytokines and growth factors that trigger local inflammation. The final outcome of these macrophage-mediated actions often results in tissue destruction. Indeed, in septic arthritis activated macrophages are early found in the synovial tissue and their numbers increase during the destructive process of inflammation [6,19]. The host must balance delicately the protective role of macrophages against their potential to cause injury and destruction of host tissues.

In the study mice rendered monocytopenic and inoculated with S. aureus developed clinically and histopathologically a less severe arthritis compared to the controls. Upon stimulation by S. aureus and its products, macrophages synthesize and release the proinflammatory cytokines TNF_{.}- and IL-6 [4,6]. Depletion of monocytes resulted in decreased production of IL-6 and TNF_{.}- as shown both in Vivo, ex vivo, and in vitro. This outcome is probably the key factor behind the downregulation of arthritis. In this respect it has been previously shown that TNF- influences directly the migration of monocytes and lymphocytes into the synovium through its effect on endothelial cell expression of intercellular adhesion molecule-1 (ICAM-1), vascular cellular adhesion molecule-1 (VCAM-1) and E-selectin [5], and indirectly through induction of chemokines such as IL-8 and monocyte chemotactic protein I (MCP-1) [12]. The detrimental role of macrophages in aseptic chronic joint diseases has previously been firmly established. Synovial macrophages play a critical role in the pathogenesis of joint erosions [2,25]. Macrophage-derived cytokines like TNF_{.}- and IL-1 dominate the cytokine profile in inflammatory synovitis [13,17]. Also in the case of septic arthritis TNF_{.}- plays a detrimental role as recently demonstrated [15]. Similar properties are shown by IL-6, a piciotropic cytokine involved in the regulation of immune responses (Hultgren, unpublished). Interestingly, the number of macrophages in synovial tissue was not grossly affected by etoposide treatment. Having in mind the significant amelioration of inflammatory cytokines it may joint pathology together with a decrease of secretion of proinflammatory cytokines it may be hypothesized that etoposide while deleting peripheral blood monocytes, arrests differentiation (e.g. production of tissue-destructive cytokines) of tissue bound macrophages. The monocytopenic mice displayed a more pronounced weight loss and a higher mortality than the controls, even when a suboptimal dose of bacteria was used. The lack of phagocytizing, monocytes resulted in a higher bacterial load in kidneys and in blood in the dei etoposide-treated animals. The lower number of peripheral granulocytes in the etoposidetreated mice as compared to the controls may also have contributed to the development of septicemia in these mice [24].

Altogether, the results indicate that monocytes/macrophages have a dual role in the development of S. aureus induced arthritis and sepsis. On one hand they contribute to tissue lesions in the joints but on the other they protect the host by more efficient elimination of bacteria.

Study 2

Collagen induced arthritis (CIA) is the most commonly used model of rheumatoid arthritis (RA). In both CIA and RA there is an increase in the cellular content of the synovium being dominated by macrophages. We decided to assess the impact of etoposide, a topoisomerase II antagonist known to induce monocyte apoptosis, on the development of CIA.

Methods.

Mice were primed and booster-immunized with collagen II. One group of mice was treated with etoposide two days prior to immunization with collagen and then on four consecutive days weekly until the end of the experiment. The second group of mice was injected with etoposide four days per week starting 40 days after collagen priming. The third group of mice were controls receiving PBS. The mice were examined concerning development of arthritis, numbers of circulating leukocytes, serum collagen II antibody and cytokine levels. Results: None of the mice administered etoposide prior to collagen immunization developed arthritis. Serum levels of anti-collagen type II antibodies were undetectable in these mice, while they displayed significantly increased levels of IFN-γ, IL-6 and MIP-1α. In addition, the collagen II specific B cell responses in the draining lymph nodes were highly suppressed. Also mice treated with etoposide at the onset of clinical arthritis showed reduced frequency of their disease by 50%.

### CONCLUSION

Study 2 demonstrates a striking disease alleviating impact of topoisomerase II antagonist on the course of collagen IL induced arthritis.

The model of collagen-induced arthritis (CIA) has been extensively used to elucidate the pathogenic mechanisms relevant to human rheumatoid arthritis (RA) and is widely used for the evaluation of potential antirheumatic agents. Similar to RA, CIA is characterized by massive infiltration of inflammatory cells in the synovium and hyperplasia of the synovial membranes in the joints (1). The infiltrating cells in the inflamed synovium in RA and in CIA include T cells, B cells, and macrophage population (2, 3). In RA as in CIA there is a scarcity of T cell cytokines but an abundance of cytokines and growth factors produced by macrophages and synovial fibroblasts (4, 5).

Etoposide is a cytostatic drug, acting by inhibiting the topoisomerase II function, and thereby leading to apoptosis. Etoposide has been shown to selectively deplete the monocyte population in mice and in rabbits (6, 7). We have previously shown that mice, pretreated with etoposide, developed a significantly less severe septic arthritis (8). However, in the case of septic arthritis, absence of bacterial clearance by monocytes/macrophages resulted in septicemia and increased mortality in etoposide-treated mice. The aim of the present study was to study the impact of etoposide administration on the development of CIA. Our results show that administration of suboptimal doses of etoposide led to a disease ameliorating effect. Most strikingly, treatment with etoposide two days prior to collagen II immunization completely inhibited the development of disease, as verified by both clinical and histopathological observations, in parallell with inability to raise collagen 11 antibodies. Surprisingly, these mice simultaneously displayed high levels of circulating gamma interferon (IFN-γ? and interleukin-6 (IL-6), indicating that these cytolines are not instrumental in mediating arthritis.

Materials and Methods

Mice

B10.QxDBA/1 female mice were used in all experiments. They were aged from 11-16 weeks. In all experiments the mice were age-matched. The mice were kept in the animal facility at the University of Göteborg, under standard conditions of light and temperature and fed standard laboratory chow and water ad libitum.

Induction of CIA and Treatment with Etoposide

Rat type II collagen (CII) from a chondrosarcoma was prepared by pepsin digestion and purification as described previously (9). The preparation of CII was dissolved at a concentration of 2 mg/ml in 0.1M acetic acid and stored at 4° C. Arthritis was induced by intradermal injection at the base of the tail with 50 μl of 100 μg CII emulsified with an equal volume of complete Freuid/s adjuvant (CFA; containing Mycobacterium butyricum, Difco, Detroit, Mich.). Mice were boosted with the same volume and concentrations of CII emulsified in incomplete Freund's adjuvant (IFA; Difco) 21 days after the first immunization.

Etoposide (Bristol Myers Squibb AB, Bromma, Sweden) acts by inhibition of DNA topoisomerase II function, interrupting the late S/G2 phase of the cell cycle, ultimately leading to apoptosis of the cell (10). Etoposide was diluted 1:10 in PBS (0.13 M NaCl, 10 mM sodium phosphate (pH 7.4) from a stock solution of 20 mg/ml. Fresh solution was prepared every day. A volume of 150-200 ?l corresponding to 12.5 mg/kg was injected subcutaneously. The dose of etoposide was chosen according to earlier studies (7).

Mice were primed and booster-immunized with CII. They were divided into three groups. One group of mice (n=19) was treated with etoposide two days prior to immunization with collagen and then on four consecutive days weekly until the end of the experiment 61 days later. The second group of mice (n=19) was injected with etoposide four days per week starting 40 days after collagen immunization. The third group of mice were controls (n—17), receiving treatment with PBS. Prior to the start of the experiment, six mice in each group were bled for hematological analyses.

At the time of sacrifice, 3 days after the last etoposide-administration, blood samples were obtained for hematological and cytokine analyses. Right front- and right hindpaw were removed from each mouse for histopathological analyses.

Clinical Evaluation of Arthritis

At regular time points the mice were examined concerning development of arthritis. Arthritis was defined as visible joint erythema and/or swelling of at least one joint. To evaluate the intensity of arthritis, a clinical scoring system of 0 to 3 points for each limb was used (1 point, mild swelling and/or erythema; 2 points, moderate swelling and erythema; 3 points, marked swelling and erythema). The arthritic index was constructed by adding the scores from all four limbs for each animal (11).

Histopathological Examinations

Sixty-one days after immunization all mice were sacrificed and one front- and one hind paw from each animal in all groups (etoposide-pretreated, etoposide-treated and controls) were removed. Histopathological examination was performed after routine fixation, decalcification, paraffin embedding, and staining with hematoxylin and eosin. All the slides were coded and the joints were studied with regard to synovial hypertrophy (defined as a synovial membrane thickness of more than two cell layers), pannus formation (synovial tissue overlaying the joint cartilage), and cartilage and bone destruction (loss of tissue integrity with resulting ingrowth of fibrotic tissue) (11). The severity of synovial hypertrophy and cartilage/bone destruction was scored from 0 (intact synovial, cartilage/bone tissue) to 3 (intense synovitis with total destruction of cartilage and/or bone).

Hematological Analyses

Mice were bled from the tail into heparinized tubes at different time-points. The leukocytes were counted in a hemacytometer (Toa Medical Electronics, Kobe, Japan). Fifty microliters of the heparinized blood was analyzed in a FACScan cytometer (Becton Dickinson, San Jose, Calif.) to determine the percentages of lymphocytes, granulocytes, and monocytes. The absolute numbers of different leukocyte subsets were then calculated from the total leukocyte counts.

Measurement of Immunoglobulins and Cytokine Levels

Serum levels of immunoglobulins were measured by the single radial immunodiffusion technique (12). Antiserum was purchased from Dako (Dako A/S, Denmark) and mouse immunoglobulin standards were obtained from Sigma (Sigma Chemical Co., St. Louis, Mo.).

For quantification of serfu CII antibodies, 96-well plates (Nunc, Roskilde, Denmark) were coated overnight at 4° C. with 10 μg/ml of native CII. Standards and samples were diluted in 0.5% BSA-PBS. Biotinylated F(ab/)₂ fragments of goat anti-mouse IgG (Jackson ImmunoResearch Laboratories, West Grove, Pa.) was used as secondary antibody. Development was performed using horseradish peroxidase 0.5 μg/ml and 2.5 mg of the enzyme substrate 2,2-azino-bis-(3-ethylbenzothiazoline sulfonic acid) (Sigma) per mal in citrate buffer (pH 4.2), containing 0.0075% H2O2. The absorbance was measured at 405 nm in a SPECTRAmax spectrophotometer (Molecular Devices, Calif.). IFN-γ levels in sera were quantified with an ELISA system previously described (13). Ninety-six well plates (Nunc) were coated with 2 μg/ml of rat anti-mouse IFN-γ mab (Pharmingen, San Diego, Calif.). Sera, recombinant IFN-γ (Genzyme, Cambridge, Mass.), biotinylated antibodies were all diluted in 0.05 M Tris pH 7.4+0.015 M NaCl. A biotinylated anti mouse IFN-γ antibody (2 μg/ml) (Pharmigen) was used as secondary antibody. After incubation with extravidin alkaline phosphatase 0.5 ?g/ml and alkaline phosphatase substrate 1 mg/ml (Sigma) the absorbance was measured at 405 nm. Interleukin 6 (IL-6) levels in serum and supernatants were measured by a bioassay using cell line B13.29, subclone B9, which is dependent on IL-6 for growth. B9 cells were harvested from tissue culture flasks, seeded into microtiter plates (Nunc) at a concentration of 5×10³ cells per well and cultured in complete medium. Serum and supernatants samples were added in twofold dilutions and in triplicates. Cultures were oulsed with 1 μCi of ³H-TdR (Radiochemical Centre, Amersham, UK) after 68 hours of culture, and the cells were harvested 4 hours later. Recombinant mouse IL-6 (Genzyme) was used as a standard The B9 cells were previously shown not to react with several recombinant cytokines including IL-1a, IL-1b, IL-2, IL-3, IL-5 and granulocyte/macrophage colony-stimulating factor, TNF and IFN-g. There was only a weak reactivity with IL-4 (14).

Levels of TNF, IL-1b, RANTES, and macrophage inflammatory protein (MIP)-1α were determined using sandwich ELISA from R&D Systems (Minneapolis, Minn.).

Ex Vivo Stimulation of Spleen and Lymph Node Cells for Proliferation as Well as Cytokine and Antibody Production

To evaluate the impact of etoposide on leukocyte proliferation and cytoline production, naive mice were treated with etoposide and with PBS (controls) 4 days weekly for 4 weeks. The mice were sacrificed 3 days after the last etoposide-treatment and single cell suspensions were prepared. The time points concerning etoposide treatment and sacrifice of animals were chosen to mimic the conditions prevailing in the experiment when the mice were immunized with CIL

The preparation of spleen mononuclear cells was performed as previously described (15). Briefly, spleen mononuclear cells from etoposide-treated mice and controls were isolated and suspended at a concentration of 1×106/ml in 24 well culture dishes (Nune) in Iscove's medium (Gibco, Paisley, UK), supplemented with 10% heat inactivated fetal calf serum (FCS) (Itegro, Leuvenheim, the Netherlands), L-glutarine, mercaptoethanol and gentamycin. The cell suspensions were incubated with 1.25 mg/ml Concanavain A (ConA) (ICN Biochemicals, Cleveland, Ohio) or 10 mg/ml of highly purified TSST-1 (Toxin Technology, Madison, Wis.). After 24 h of incubation at 37° C. in 5% CO2, the cell culture supernatants were collected and kept at −20° C. until analysis. Proliferation assay was performed in the same way in 96-well microtiterplates (Nunc) and cultured for 72 h. During the last 12 h 1 mCi ³H-thymidine/well (Radiochemical Centre, Amersham, UK) was added. To test the effect of etoposide on the immune response of draining lymph nodes, mice were immunized with C-U emulsified in CFA (1 mg CII/ml, 50 ml/mouse) in both hindpaws and base of the tail. One group of mice (n=3) was treated with etoposide two days prior to immunization with collagen and then on four consecutive days until the sacrifice day 10. The second group of mice were controls (n=3), receiving treatment with PBS. On day 10, the mice were sacrificed and draining (popliteal and inguinal) lymph nodes were taken for FACS analysis, immunohistochemical examination and stimulated with CII in cell culture assays. Supernatants from lymph node cells incubated with CII in vitro were evaluated by ELISA for CII antibodies.

In Vitro Chemokine-and Cytokine Release.

In order to study the influence of etoposide on macrophages, IC-21 cells, a murine macrophage cell clone (ATCC TIB 186) was used. These cells display many typical features of the macrophage phenotype and are therefore a suitable model for studying cytoline and chemokine release during inflammation (16). The cells were maintained in continous culture using RPMI-1640 supplemented with 10% FCS, L-glutamine, and gentamycin. When the cells had grown to about 70% confluency, they were seeded into a 24-well-plate (Nunc) at a concentration of 1×10⁵ per well and incubated at 37° C. in 5% CO2 for 24 h. After 24 h the medium was changed into etoposide-containing medium at concentrations 0.1 and 0.01 mM. These concentrations of etoposide were found in preliminary experiments not to influence the cell viability or total cell numbers (data not shown). Furthermore, concentrations of etoposide lower than 0.1 μM have been shown not to affect the cell number of L 929 cell clone (17). After 24 h of incubation, the cells were washed once with PBS, and reincubated with medium containing different concentrations (1, 10, 100, 1000 ng/ml) of LPS (Difco) and IFN-γ (10, 50, 100 u/nml). The interferon-γ was purified from the supernatants from Chinese hanater ovary (CHO) cells transfected with the murine IFN-γ gene (hybridoma kindly provided by Dr. Morris, Department of Biological Sciences, University of Warwick, Coventry, UK). After 2 and 24 h of incubation the supernatants were collected and stored at −20° C. until analysis.

Statistical Analysis

Statistical comparisons were made by the Mann-Whitney U test and chi-square test with Yates correction. All values are reported as the mean±standard error of the mean (SEM).

Results

Mice Pretreated with Etoposide are Resistant to Collagen II Induced Arthritis

Administration of etoposide two days prior to immunization with CIE completely inhibited induction of the disease. Throughout the course of the experiment (almost nine weeks) none of 19 mice pretreated with etoposide (“Etoposide-pretreated”) displayed any clinical signs of arthritis (FIG. 5, Table 5). Mice that received etoposide 40 days after collagen immunization (“Etoposide-treated”) developed arthritis with a lower incidence than the controls. At the end of the experiment 29% (5 of 17) of the etoposide-treated mice exhibited arthritis versus 53% (9 of 17) in the control group (FIG. 5). No significant changes in weight between or within the groups could be observed.

The histopathologic analyses confirmed the clinical findings. None of the etoposide pretreated mice displayed synovial hypertrophy or bone/cartilage destruction. In contrast, significant differences were not observed between the group of mice treated with etoposide after immunization and the controls (Table 6).

Levels of Cytokines, Chemokines and Immunoglobulins in Sera of Etoposide Treated Mice

Sera from CII immunized mice were collected and analyzed at the end of the experiment. Levels of IL-6 and IFN-γ in sera from mice treated with etoposide prior to, or after, CII immunization were significantly higher as compared to control animals (Table 7). Interestingly, mice pretreated with etoposide did not produce CII antibodies (Table 7). Both groups that received etoposide displayed decreased total levels of serum immunoglobulins as compared to the controls (etoposide pretreated 9.2±0.5 mg/ml, etoposide treated 8.5±0.7 mg/ml, controls 13.2±0.8 mg/ml; p<0.001 versus controls). Levels of MIP-1α in serum were very low in all three groups. However, the animals that received etoposide displayed significantly higher levels as compared to the controls (Table 7).

Hematological Analyses

The CII immunized mice were exsanguinated 61 days after immunization, i.e. three days after the last etoposide administration. Surprisingly, mice treated with etoposide displayed significantly higher numbers of peripheral lymphocytes, granulocytes and monocytes compared to the control animals (Table 8).

In another experiment etoposide was administered to healthy, non-immunized mice and the mice subsequently sacrificed at time points that mimiced those used in the experiment with CE immunized mice. Thus, naive mice were injected with 12.5 mg/kg of etoposide 4 days per week for 4 weeks. Peripheral blood cell count was performed 3 days after the last etoposide treatment. Even here, there was a tendency to higher numbers of monocytes and granulocytes in the mice treated with etoposide versus controls treated with PBS ([71±28]×10⁴/ml versus [58±4.2]×10⁴/ml for monocytes and [1.1±0.4]×10⁴/ml versus [0.7±0.1]×10^{4/ml for granulocytes).}

To investigate whether etoposide affected the draining lymph node response, known to precede the onset of CIA, the inguinal lymph nodes were isolated 10 days after CII immunization. Surprisingly, no significant effects on numbers of B cells (CD 19, B220, MHC class H), T cells (CD4, CD8) or macrophages (CD11b, F4/80) or formation of germinal centers were seen using immunohistochemical stainings of inguinal lymph nodes. In addition, no effect on T or B cell populations using flow cytometric analysis was noted. However, challenge of lymph node cells in vitro with CII revealed a dramatic decrease of B cell functions since antibodies to CII were not produced at all in cell cultures from etoposide treated mice in contrast to PBS controls whereas no difference in T cell functions such as proliferation or IFN-γ secretion were seen (data not shown).

The Impact of Etoposide on Ex Vivo Proliferative Responses and Cytokine Production in Naive Mice

Spleen cells from naive mice, treated with etoposide for 4 days per week during 4 weeks, displayed a significantly lower proliferative response to TSST-1 and Con A as compared to the controls (Table 9). Furthermore, treatment with etoposide significantly downregulated production of IL-6 and TNF by spleen cells from etoposide-treated mice (Table 9). Since IL-6 and TNF are predominantly secreted by macrophages these results indicate that etoposide may modify the function of this cell population.

Etoposide Influences the Release of Chemokines and Cytokines

To investigate whether etoposide influences the ability of monocytes/macrophages to release chemokines and cytokines, a murine macrophage cell line was incubated with etoposide at concentrations previously shown not to induce apoptosis. After incubation with etoposide, the cells were stimulated with different concentrations of LPS or IFN-γ. It was clear that etoposide at the concentration 0.1 μM down-regulated the production of TNF following stimulation with LPS and with IFN-γ (Table 10). In addition, also IFN-??triggered MIP-1α release was decreased upon treatment with etoposide (Table 11). The levels of IL-6 were downregulated by etoposide when stimulation was performed by LPS (FIG. 6A). When stimulated with IFN-γ levels of IL-6 were very low but seemed to be upregulated by etoposide (FIG. 6B). The chemokine RANTES was found to be upregulated by etoposide (data not shown). No major differences could be noted with respect to levels of IL-1α.

Discussion

In this study we treated mice with suboptimal doses of the monocyte-depleting agent etoposide prior to and after immunization with type II collagen (CII). Administration of etoposide prior to CII immunization had a most striking effect on the development of arthritis. Indeed, none of these mice displayed any signs of arthritis, neither clinically nor histopathologically. In parallell with this finding antibodies to collagen II were not detectable. A strong antigen-specific B cell response is operational in CIA (18). B cells appears to be required for the induction of disease since B cell-deficient mice do not develop CIA (19). More importantly, CII specific antibodies are important effector molecules in collagen II arthritis, as shown by passive transfer experiments (20. 21, 22).

Thus, one of the major reasons for the beneficial results obtained in the present study is the total absence of CII specific B cell responses. This outcome might be due to either a general suppression of immune responses or to effects more specifically directed to B cells. In this respect it is important to study the general B cell reactivity during treatment with etoposide.

The complete lack of antibodies to CII in sera of etoposide treated mice is a remarkable finding. Taken together the lack of induced antibody production in lymph node cells but normal B cell numbers argues for a directed effect on B cell function. Having in mind the relatively modest decrease of total immunoglobulin production following long-term etoposide treatment it is suggested that the drug is a highly potent inhibitor of newly activated B lymphocytes.

We and others have previously shown that etoposide selectively deplets the monocytic cell population in rabbits (23) and in mice (6, 8). Thus, earlier findings have mainly implicated macrophages as the main target for etoposide in modulating effects on inflammatory diseases. In the present experiments we could not confirm a depletion of monocytes/macrophages in blood and in draining lymph nodes. In fact, at the end of the experiment we found higher levels of circulating leukocytes than in the controls as well as higher serum levels of IFN-γ and IL-6, cytokines suggested to be proinflammatory in CIA. However, etoposide may modulate monocyte functions, since spleen cells from etoposide treated mice displayed lower IL-6 and TNF production as compared to the controls. In addition, in vitro etoposide treated macrophages showed reduced capacity to produce TNF after stimulation with IFN-γ.

Both pro-and anti-inflammatory properties have been ascribed to IL-6 and IFN-γ?. There are studies reporting IFN-γ ?to be disease-promoting in CIA (24, 25). In contrast, some other studies present a protective effect of IFN-γ?in CIA (26, 27). Also in RA, treatment with IFN-γ proved to be beneficial (28). There are several suggestions of how IFN-γ ?acts as a disease-limiting factor. It has been shown in vitro that IFN-γ?inhibits metalloproteinase production and glycosaminoglycan release by cultured cartilage fragments (29). In another in vitro experiment, IFN-γ?exerted an inhibitory effect on bone resorption.?Furthermore??monocytes are induced by IFN-γ to produce TGF-α, which is known as an anti-inflammatory cytokine (30). Finally, IFN-γ down-regulates the levels of the proinflammatory chemokine MIP-1α, as demonstrated in vitro in this study and previously shown by Horton and coworkers (31). MIP-1α and RANTES are chemokines with potent inflammatory effects in RA, mainly mediated by the recruitment of primarily mononuclear cells into the joint (32, 33). Messenger RNA specific for MIP-1α and RANTES is elevated both early and late in CIA, suggesting that these chemokines play a role throughout the course of the disease (34). Interleukin-6 is present at very high levels in serum and synovial fluids of patients with RA (4) and has been suggested to play an important role in the development of CIA (35). However, other studies have shown that administration of IL-6 suppressed the DTH response to SRBC and treatment with IL-6 ameliorated the development of adjuvant arthritis in rats (36).

Taken together, the most striking effect on immune functions in our experiments is the dramatic effect on the B cell response to CII. Together with the fact that CIA is largely mediated by CII antibodies binding to cartilage and thereby triggering an immune complex mediated arthritis it is likely that the main effect of etoposide on CIA is mediated by directly or indirectly suppressing B cell functions. However, this does not exclude effects on monocyte/macrophages or other inflammatory cells. In fact, previous therapeutic effects by etoposide using the septic arthritis model, that is not mediated by arthritogenic antibodies, highlight alternative possibilities. It is possible that modulation of macrophage activity may regulate the B cell activation to CH since macrophages, rather than dendritic cells, are the main priming antigen presenting cells for CII reactive T cells (37). Clearly B cells play an important role in RA as the production of rheumatoid factor is one of the hallmarks of the disease. However, a direct role of arthritogenic antibodies has not been excluded and there are recent reports suggesting that arthritogenic anti-CII antibodies reactive with identical epitopes in the mouse, rats and humans are produced (38, 39, 40).

Taken together, the present study demonstrates a beneficial impact of etoposide treatment on the course of collagen II induced arthritis. Based on above findings we believe that clinical trials with etoposide treatment in RA patients would be of interest.

The test data obtained shows that the present invention will have an effect in the treatment of inflammations including arthritis, as well as asthmatic conditions, ulcerous colitis, and connecting tissue inflammations in humans as well.

A second aspect of the invention is that the amount of etoposide administered is only ¼ to ½ of the amount of etoposide used as a cytostatic agent. Thus the severe side effects occurring when using etoposide as a cytostatic can be substantially eliminated. Thus the daily dose of etoposide for treatment according to the present invention is 0.2-0.7 mg/kg bodyweight, compared to 0.75-1.25 mg/kg bodyweight when used as a cytostatic agent.

A further feature of the invention is to obtain a combination of etoposide and an antibiotic agent, particularly at the treatment of septic arthritis, whereby the daily dose of antibiotic agent is dependent upon the recommended dose of the particular antibiotic agent selected, normally 50 to 2000 mg per 24 hrs, or 1 to 40 mg per kg body weight and 24 hrs.

The compounds of the present invention can be administered in the form of oral, rectal, injection, or inhalation preparations. Oral compositions normally exist as tablets, granules, capsules (soft or hard), or powders, either coated or uncoated products. As coated products they may be merely enteric coated to provide for a more readily administered preparation, or as a sustained release coated composition, where the release of active compound will take place due to the dissolution of the coating, which dissolution is dependent on where in the gastrointestinal tract one will have a release. Thus the release can be controlled as to place and time. It may also be advantageous to coat the active compound if this is subject to degradation, such as by gastric acid, in order then to have the compound to pass the stomach.

Tablets and capsules normally contain one dose of the active compound, i.e., the dose determined to fulfil the requirements of obtaining a therapeutically active level in serum or otherwise, either this is required once, twice or more times a day (24 hrs).

Rectal compositions are normally prepared as suppositories, where the active compound is dissolved or dispersed in a waxy compound or fat having a melting temperature in the range of the body temperature, as to release the active compound when administered rectally.

Preparations for injection are commonly made for subcutaneous, intramuscular, intravenous, or intra peritoneal administration. Injection solutions are normally provided with an adjuvant to facilitate absorption of the active compound.

Preparations for inhalation are commonly present as powders which are administered either in pressurized containers with a dosing nozzle, or in an inhaler system where the powder is dosed in the system and then the patient is inhaling air through the apparatus to such degree that the powder becomes airborne and enters the respiratory tract, including the lungs. Inhalation preparation are normally used for inflammatory conditions in the respiratory tract including the lungs.

The compositions contain 0.5 to 99% by weight of active compound, and the remainder is different inert, non-therapeutically active compounds which facilitate administration, preparation such as granulation, tableting, or storage. Such inert materials may, however, have a administratively positive effect.

The active compound of the invention, etoposide, is administered in an amount of 1 to 100 mg per kilogram body weight depending on the condition of the patient, route of administration, age and body weight of the patient, and other considerations made by the physician. The most important aspect hereby is the serum concentration which may be 0.1 to 100 mM of active compound, in accordance with the present findings.

### REFERENCES

Study 1

1. Abdelnour, X, S. Arvidson, T. Bremell, C, Rydén, and A. Tarkowski. 1993.

The accessory gene regulator (agr) controls Staphylococcus aureus virulence in a murine arthritis model. Infect. Immun. 61: 3879-3885.

2. Abdelnour, A., T. Bremell R. Holndahl, and A. Tarkowski. 1994.

Clonal expansion of T lymphocytes causes arthritis and mortality in mice infected with toxic shock syndrome toxin-1-producing staphylococcal. Eur. J. Immunol. 24: 1161-1166,

3. Abdelnour, A, T. Bremell, R. Holhndahl, and A. Tarkowski. 1994. Role of T lymphocytes in experimental Staphylococcus aureus arthritis. Scand. S. Immunol, 39:403-408.

4. Abdelnour, A., and A, Tarkowski. 1993.

Polyclonal B-cell activation by an arthritogenic Staphylococcus aureus strain: contribution of T-cells and monokines. Cell. Immunol, 147: 279-293.

5. Bevilacqua, M. P., R. M. Nelson, G. Mannori, and O, Cecconi, 1994.

Endothelial-leukocyte adhesion molecules in human disease. Annu. Rev, Med. 45: 361-378.

6. Bremell, T., A. Abdelnour, and A. Tarkowski, 1992.

Histopathological and serological progression of Staphylococcus aureus arthritis. Infect. Immun. 60:2976-2985.

7. Bremeli, T, 5. Lange, R. Holndahl, C. Ryde'n, G. L Hansson, and A. Tarkowski.1994. Immunopathological features of rat Staphylococcus aureus arthritis, infect. Immun. 62: 233-2344.

8. Bremell, T., S. Lange, L. Svensson, E. Jenische, K, Grondahl, H. Carlsten, and A. Tarkowski. 1990.

Outbreak of spontaneous staphylococcal arthritis and osteitis in mice. Arthritis. Rheum, 33: 1739-1744.

9. Bremell, T, S. Lange, A. Yacob, C. Ryde'n, and A, Tarkowski. 1991

Experimental Staphylococcus aureus arthritis in mice. Ifect. Immun. 59: 2615-2623.

10. Calarne, W., A. E. Douwes-Idern, M, T. van den Barselaar, R. van Furth, and H. Mattie. 1994. Influence of cytostatic agents on the pulmonary defence of mice infected with Klebsiella pneumoniae and on the efficacy of treatment with cefiriaxone, J. Infect, 29-53-66.

11. Caristen, H, L-A. Nilsson, and A. Tarkowski. 1986.

Impaired cutaneous delayed type hypersensitivity in autoimmune MRL 1 pr/1 pr mice. Int. Archs. Allergy. Appl. Immun. 81.22-325.

12. Cutolo, M., A. Sulli, A. Barone, B, Seriolo, and S. Accardo. 1993.

Macrophages, synovial tissue and rheumatoid arhritis. Clin. Exp. Rheumatol. 11: 331-339.

13. Figler, A., B. Sinha, G. Hartmann, and S. Endres. 1997. Taming TNF: strategies to restrain this proinflammatory cytokine. Immunol. Today. 18: 487492.

14. Helle, M., L. Boeije, and L. A. Aarden. 1988.

Functional discrimination between interleukin-6 and interleukin-1. Eur. J. Immunol. 18:1535-1540.

15, Hultgren, O, H. P. Eugster, J. D, Sedgwick, H. Körner, and A. Tarkowski, 1998. TNF/Lymphotoxin_{.}- double-mutant mice resist septic arthritis but display increased mortality in response to Staphylococcus aureus. J. Immunol. 161: 5937-5942.

16. Johnson, R-A. Tarkowski, and L. Klareskog.

A demineralization procedure for immunohistopathological use: EDTA treatment preserves lymphoid cell surface antigens. J. Immunol. Meth.88: 109-114.

17. Jorgensen, C- and S. Gay. 1998.

Gene therapy in osteoarticular diseases: where are we? Immunol. Today. 19: 387-391.

18. Josefsson, E. H. Carlsten, and A. Tarkowski. 1993.

Neutrophil mediated inflammatory response in murine lupus. Autoimmunity. 14: 251-257.

19. Peters, K. M., K. Koberg, T. Rosendahl, B. Klosterhalfen, A. Straub, and G. Zwadlo-Klarwasser. 1996.

Macrophage reactions in septic arthritis. Arch. Orthop. Trauma. Surg. 115: 347-350.

20. Smith, P. L. S. Soues, T. Gottlieb, S. J. Falk, J. V. Watson, R. J. Osborne, and N. M. Blechen. 1994.

Etoposide-induced cell cycle delay and arrest-dependent modulatic a of DNA topoisomerase II in small-cell lung cancer cells. Br. J. Cancer. 70: 914-921.

21. Thörig, L., J. Thompson, F. Eulderink, J. J. Emeiss, and R. van Furth. 1980.

Effects of monocytopenia and anticoagulation in experimental Streptococcus sanguis endocarditis. Br. J. Pathol. 61: 108-116.

22. van den Berg, W. B., and P. L. E. M. van Lent. 1996.

The role of macrophages in chronic arthritis. Immunobiol. 195: 6 4-623.

23. van't Wout, J. W. I. Linde, P. C. J. Leijh, and R van Furth. 1989.

Effect on irridiation, cyclophosphamide, and etoposide (VP-16) on number of peripheral leukocytes in mice under normal conditions and during acute inflammatory reaction. Inflammation 13: 1-14.

24. Verdrengh, M., and A. Tarkowski. 1997.

Role of neutrophils in experimental septicemia and septic arthritis induced by Staphylococcus aureus. Infect. Immun.65: 2517-2521.

25. Yanni, G. A. Whelan, C. Feighery C. and B. Bresnihan 1994.

Synovial tissue macrophages and joint erosion in rheumatoid arthritis. Annals. Rheurn. Dis. 53:39-44.

26. Zhao, Y-X., A. Abdelnour, R. Holmdall, and A. Tarkowsli. 1995.

Mice with the xid B-cell defect are less susceptible to developing Staphylococcus aureus induced arthritis. J. Immunol. 155: 2067-2076.

27. Zhao, Y-X., A. Abdelnour, Å. Ljungdahl, T. Olsson, and A. Tarkowski. 1995.

Patterns of interferon_{.}- mRNA expression in toxic shock syndrome toxin-1 expanded V11⁺ T lymphocytes. Cell. Immunol. 161: 28-33.

28. Zhao, Y-X., A. Ljungdahl, T. Olsson, and A. Tarkowski. 1996.

In situ hybridization analysis of synovial and systemic cytoline mRNA expression in superantigen-mediated Staphylococcus aureus arthritis. Arthritis. Rheum. 39: 959-967.

Study 2

1. Hohldahl R, Andersson M, Goldschmidt T J, Gustafsson K, Jansson L, Mo J A. Type II collagen autoimmunity in animals and provocations leading to arthritis. Immunol Rev 1990; 118: 193-232.

2. Bresnihan B. The synovial lining cells in chronic arthritis. Br J Rheumatol 1992; 31: 433-5.

3. Holmdahl R, Tarkowski A, Jonsson R.

Involvement of macrophages and dendritic cells in synovial inflammation of collagen induced arthritis in DBA/1 mice and spontaneous arthritis in MRL/Lpr mice. Autoimmunity 1991; 8: 271-80.

4. Firestein G S, Alvaro-Gracia M, Maki R, Alvaro-Garcia J M.

Quantitative analysis of cytokine gene expression in rheumatoid arthritis. J Immunol 1990; 144: 3347-53.

5. Burmester G R, Stuhllnuller B, Keyszer G, Kinne R W.

Mononuclear phagocytes and rheumatoid synovitis. Mastermind or workhorse in arthritis? Arthritis Rheum 1997; 40: 5-18.

6. van't Wout J W, Linde I, Leijh P C J, van Furth R.

Effect of irradiation, cyclophosphamide, and etoposide (VP-16) on number of peripheral leukocytes in mice under normal conditions and during acute inflammatory reaction. Inflammation 1989: 13: 1-14.

7. Calame W, Douwes-Idema A E, van den Barselaar M Th, van Furth R, Mattie H. Influence of cytostatic agents on the pulmonary defence of mice infected with Kebsiella pneumoniae and on the efficacy of treatment with ceftriaxone. J Infect 1994; 29: 53-66.

8. Verdrengh M, Tarkowsli A.

Role of macrophages in Staphyloccus aureus induced arthritis. Arthritis Rheumn 2000; 43: 2276-82.

9. Andersson M, Holmdahl R.

Analysis of type II collagen-reactive T cells in the mouse. I. Different regulation of autoreactive vs. non-autoreactive anti-type II collagen T cells in the DBA/1 mouse. Eur J Immunol 1990; 20: 1061-6.

10. Smith P J, Soues S, Gottlieb T, Falk S J, Watson J V, Osborne R J, et al.

Etoposide-induced cell cycle delay and arrest-dependent modulation of DNA topoisomerase II in small-cell lung cancer cells. Br J Cancer 1994; 70: 914-21.

11. Abdelnour A, Arvidson S, Bremell T, Ryde'n C, Tarkowski A.

The accessory gene regulator (agr) controls Staphylococcus aureus virulence in a murine arthritis model. Inect Immun 1993; 61: 3879-85.

12. Mancini G, Carbonara A O, Heremans J F.

Immunochemical quantitation of antigens by a single radial immunodiffusion Immunochemistry 1965; 2: 235-54.

13. Zhao Y X, Tarkowski A.

Impact of interferon-γ receptor deficiency on experimental Staphylococcus aureus septicemia and arthritis. J Immunol 1995; 155: 5736-42.

14. Helle M, Boeije L, Aarden L A.

Functional discrimination between interleukin-6 and interleukin-1. Eur J Immunol 1988; 18: 1535-40.

15. Zhao Y X, Abdelnour Å, Ljungdahl A, Olsson T, Tarkowsld A.

Patterns of interferon-γ mRNA expression in toxic shock syndrome toxin-i expanded V_{β}11⁺ T lymphocytes. Cell Immunol 1995; 161: 28-33.

16. Tu B, Wallin A, Moldeus P, Cotgreave ??.

Direct exposure to nitrogen dioxide fails to induce the expression of some inflammatory cytokines in an IC-21 murine macrophage cell model. Toxicology 1995; 104: 159-64.

17. Bonellh G, Sacchi M C, Barbiero G, Duranti F, Goglio G, Verdun di Cantogno L, et al. Apoptosis of L929 cells by etoposide: a quantitative and kinetic approach. Exp Cell Res 1996; 228: 292-305.

18. Stuart J M, Tomoda K, Yoo T J, Townes A S, Kang A H.

Serum transfer of collagen-induced arthritis. II. Identification and localization of autoantibody to type II collagen in donor and recipient rats. Arthritis Rheum 1983; 26: 1237-44.

19. Svensson L, Jirholt J, Hobndahl R, Jansson L.

B cell-deficient mice do not develop type II collagen-induced arthritis (CIA). Clin Exp Immunol 1998; 111: 521-6.

20. Stuart J M, Dixon F J.

Serum transfer of collagen-induced arthritis in mice. J Exp Med 1983; 158: 378-92.

21. Wooley P H, Luthra H S, Krco C J, Stuart J M, David C S.

Type II collagen-induced arthritis in mice. II. Passive transfer and suppression by intravenous injection of anti-type II collagen antibody or free native type II collagen. Arthritis Rheum 1984; 27: 1010-7.

22. Holndahl R. Jansson L, Larsson A, Jonsson R.

Arthritis in DBA/1 mice induced with passively transferred type II collagen immune serum. Immunohistopathology and serum levels of anti-type II collagen auto-antibodies. Scand J Immunol 1990; 31: 147-57.

23. Thörig L, Thompson J. van Furth R.

Effect of immunization on the induction and course of experimental Streptococcus sanguis and Staphylococcus epidermidis endocarditis. Infection 1980; S: 267-74.

24. Cooper S M, Sriram S, Ranges G E.

Suppression of munine collagen-induced arthritis with monoclonal anti-Ia antibodies and augmentation with IFN-gamma. J Immunol 1988; 141: 1958-62.

25. Mauritz N J, Holndahl R, Jonsson R, van der Meide P H, Scheynius A, Klareskog L. Treatment with gamma-interferon triggers the onset of collagen arthritis in mice. Arthr Rheum 1988; 31: 1297-1304.

26. Nakajima H, Takamori H, Hiyama Y, Tsukada W.

The effect of ttreatment with interferon-gamma on type II collagen-induced arthritis. Clin Exp Immunol 1990; 81: 441-5.

27. Vermeire K Heremans H, Van de Putte M, Huang S, Billiau A, Matthys P.

Accelerated collagen-induced arhritis in IFN-g receptor-deficient mice. J Immunol 1997; 158: 5507-13.

28. German Lymphokine Study Group [editorial].

Double blind controled phase III multicenter clinical trial with interferon gamma in rheumatoid arthritis. Rheumatol Int 1992, 12: 175-85.

29. Andrews H J, Bunning R A, Dinarello C A, Russell R G.

Modulation of human chondrocyte metabolism by recombinant human interferon gamma: in-vitro effects on basal and IL-1-stimulated preoteinase production, cartilage degradation and DNA synthesis. Biochem Biophys Acta 1989; 1012: 128-34.

30. Billiau A- Interferon-? in autoimmunity.

Cytokine & Growth Factor Reviews 1996; 7: 25-34.

31. Horton M R, Burdick M D, Strieter R M, Bao C, Noble P W.

Regulation of hyaluronan-induced chemokine gene expression by IL-10 and IFN-γ in mouse macrophages. J Immunol 1998; 160: 3023-30.

32. Koch A E, Kunkel S L, Harlow L A, Mazaraids D D, Haines G K, Burdick M D, et al. Macrophage inflammatory protein-1 alpha. A novel chemotactic cytoldne for macrophages in rheumatoid arthritis. J Clin Invest 1994; 93: 921-S.

33. Volin M V, Shah M R, Tokuhira M, Haines III G K, Woods J M, Koch A E.

RANTES expression and contribution to monocyte chemotaxis in arthritis. Clin Immunol Immunopathol 1998; 89: 44-53.

34. Thornton S, Duwel L E, Boivin G P, Ma Y, Hirsch R.

Association of the course of collagen-induced arthritis with distinct patterns of cytokine and chemokine messenger RNA expression. Arthritis Rheum 1999; 42: 1109-18.

35. Sasai M, Saeki Y, Ohshima S, Nishioka K, Mima T, Tanaka T, et al.

Delayed onset and reduced severity of collagen-induced arthritis in interleukin-6-deficient mice. Arthritis Rheum 1999; 42: 1635-43.

36. Mihara M, Ikuta M, Koishihara Y, Ohsugi Y.

Interleukin 6 inhibits delayed-type hypersensitivity and the development of adjuvant arthritis. Eur J Immunol 1991; 21: 2327-31.

37. Michalesson E, Hohndahl M, Engstrom A, Burkhardt H, Scheynius A, Holmdahl R. Macrophages, but not dendritic cells, present collagen to T cells. Eur J Immunol 1995; 25: 2234-41.

38. Schulte S, Unger C, Mo J A, Wendler O, Bauer E, Frischholz S. et al.

Arthritis-related B cell epitopes in collagen II are conformation-dependent and sterically pivileged in accessible sites of cartilage collagen fibrils. J Biol Chem 1998; 273: 1551-61.

39. Kraetsch H G, Unger C, Werrhoff P, Schneider C, Kalden J R, Hohndahl R, et al. Persistance of a cartilage-specific autoantibody response in rheumatoid arthritis: characterization of a conformational B cell epitope in the integrin-binding domain of collagen type II. Eur J Immunol 2001; in press.

40. Wernhoff P, Hofmann C, Bajtner E, Burkhardt H, Holmdatl R. B cell recognition of type II collagen in the rat. Int Immunol 2001; in press.

TABLE 1
Growth of S. aureus in kidneys and blood of mice pretreated
with etoposide three days prior to injection with
1˜2 × 10⁷ CFU of S. aureus LS ˜ I^{a}.
Bacterial growth in kidney (× 10⁷ CFU)
Bacterial growth in blood (× 10⁷ CFU/ml)

| Day | Etoposide | n | Controls | n | Etoposide | n | Controls | n |
|---|---|---|---|---|---|---|---|---|
| 3 | 3 ± 2 | 5 | 2 ± 15 | 4 | 60 ± | 10 | 100 ± 60 | 10 |
| | | | | | 130^{b} | | | |
| 6-7 | 67 ± 12 | 17 | 45 ± 8 | 21 | 290 ± 100 | 16 | 250 ± 130 | 22 |

^{a}The number of colony forming units (CFU) in homogenates of two kidneys and blood from each animal were determined on blood agar plates. The results are expressed as mean + SEM
^{b}p < 0.01 in comparison with control

TABLE 2
Serum cytokine levels in etoposide-treated
mice (n = 6) and controls (n = 8) six days
after injection with 1.4 × 10⁷ CFU of S. aureus.^{a}

| | |
|---|---|
| IL-6 (pg/ml) | TNF-α (pg/ml) |

| Etoposide | Controls | Etoposide | Controls |
|---|---|---|---|
| 11.927 ± 4.610^{b} | 23.867 ± 5.059 | 30 ± 12 | 57 ± 10 |

^{a}mice were pretreated with 12.5 mg/kg of etoposide three days prior to bacterial inoculation. Data are presented as the mean ± SEM.
^{b}p < 0.05 in comparison with the controls

TABLE 3
In vitro cytokine production by spleen
mononuclear cells from non-infected mice.
The cells were incubated with different
concentrations of etoposide and stimulated
with TSST-1 and Con A^{a}

| | IL-6 (pg/ml) | | TNF.- (pg/ml) | |
|---|---|---|---|---|
| Etoposide (μM) | ConA | TSST-I | ConA | TSST-1 |
| 0 | 988 ± 429 | 273 ± 27 | 180 ± 47 | 128 ± 39 |
| 1 | 825 ± 455 | 45 ± 16b | 167 ± 29 | 64 ± 20 |
| 10 | <10^{b} | <10^{b} | 66 ± 19^{b} | 38 ± 8^{b} |
| 100 | <10^{b} | <10^{b} | 10 ± 1^{b} | 16 ± 3^{b} |

^{a}the data are expressed as mean ± SEM from four mice
^{b}p < 0.05 vs etoposide 0 μM

TABLE 4
Cytokine production by spleen
mononuclear cells from non-infected,
in vivo etoposide-treated mice.
The cells were stimulated with
TSST-1 and Con A^{a}

| | IL-6 (pg/ml) | | TNF- (pg/mI) | |
|---|---|---|---|---|
| | ConA | TSST-I | ConA | TSST-I |
| Etoposide-treated | 455 ± 85^{b} | 255 ± 66b | 138 ± 17 | 77 ± 22 |
| Controls | 1225 ± 185 | 495 ± 87 | 227 ± 40 | 104 ± 25 |

^{a}the data are expressed as mean ± SEM from four nuice per group
^{b}p < 0.05 vs controls

TABLE 5
Severity of CIA in etoposide treated mice*

| | Arthritic score of diseased mice | | |
|---|---|---|---|
| | mean ± SEM | | |
| Manipulation | Day 46 | Day 53 | Day 61 |
| Etoposide | 0 | 0 | 0 |
| pretreated | | | |
| Etoposide | 2.4 ± 0.2 (n = 5) | 5.4 ± 1.3 (n = 5) | 5.6 ± 1.3 (n = 5) |
| treated | | | |
| Controls | 2.4 ± 0.6 (n = 7) | 3.4 ± 0.8 (n = 10) | 4.2 ± 0.7 (n = 9) |

*Mice were treated with 12.5 mg/kg of etoposide 2 days prior to (“Etoposide-pretreated”) or 40 days after (“Etoposide-treated”) collagen II immunization. The differences are not statistically significant.

TABLE 6
Histopathologic evaluation of joints*
of mice treated with etoposide
two days prior to or 40 days after
collagen II immunization

| Manipulation | n | Synovial hypertrophy | Cartilage/bone destruction |
|---|---|---|---|
| Etoposide | 16 | 0 | 0 |
| pretreated | | | |
| Etoposide | 17 | 2.4 ± 1.1 | 4.5 ± 2.4 |
| treated | | | |
| Controls | 16 | 2.6 ± 1.2 | 3.4 ± 1.8 |

*Mean synovial hypertrophy score (mean ± SEM). Mice were treated with 12.5 mg/kg of etoposide 2 days prior to (“Etoposide-pretreated”) or 40 days after (“Etoposide-treated”) collagen immunization. One hind-and one frontpaw from each animal was examined.

TABLE 7
Serum levels of immunoglobulins, IFN-g, IL-6 and MIP-1a in mice receiving etoposide*

| | | Anti-collagenII | Total immunoglobulin | | | |
|---|---|---|---|---|---|---|
| | | antibody | levels | IFN-g | IL-6 | MIP-1a |
| Manipulation | n | (mg/ml) | (mg/ml) | (u/ml) | (pg/ml) | (pg/ml) |
| Etoposide- | 18-19 | 1 ± 0^{a} | 9.2 ± 0.5a | 1848 ± 248^{c} | 117 ± 21a | 3.0 ± 0.5^{c} |
| pretreated | | | | | | |
| Etoposide- | 17 | 29 ± 4 | 8.5 ± 0.8^{a} | 2094 ± 199^{b} | 179 ± 21^{b} | 2.9 ± 0.3^{c} |
| treated | | | | | | |
| Controls | 16-17 | 39 ± 4 | 13.2 ± 0.8 | 1202 ± 175 | 53 ± 7 | 1.9 ± 0.2 |

*Data are expressed as mean ± SEM. Mice were treated with 12.5 mg/kg of etoposide 2 days prior to (“Etoposide-pretreated”) or 40 days after (“Etoposide-treated”) collagen immunization.
^{a}p < 0.001 versus controls
^{b}p < 0.01 versus controls
^{c}p < 0.05 versus contro

TABLE 8
Total numbers of peripheral leukocytes in mice treated
with etoposide and immunized with collagen II*

| | Monocytes (× 10⁴/ml) | | Granulocytes (× 10⁴/ml) | | Lymphocytes (× 10⁶/ml) | |
|---|---|---|---|---|---|---|
| Manipula- | | | | | | |
| tion | Day-2 | Day 61 | Day-2 | Day 61 | Day-2 | Day 61 |
| Etoposide- | 18 ± 4 | 67 ± 20 | 71 ± 13 | 698 ± 341^{b} | 4.7 ± 0.3 | 7.1 ± 3.4 |
| pretreated | | | | | | |
| Etoposide- | 19 ± 3 | 92 ± 24^{a} | 87 ± 20 | 991 ± 326^{b} | 5.9 ± 0.8 | 14.3 ± 3.3^{b} |
| treated | | | | | | |
| Controls | 21 ± 3 | 30 ± 24 | 60 ± 6 | 32 ± 5 | 5.9 ± 0.5 | 5.1 ± 0.6 |

*Data are presented as mean ± SEM. Mice were injected with etoposide prior to (“Etoposide-pretreated”) or 40 days after (“Etoposide-treated”) immunization with collagen II. Blood cell populations were analyzed prior to (Day-2) and at the end of the experiment (Day 61)
^{a}p < 0.05 versus controls
^{b}p < 0.01 versus controls

TABLE 9
In vitro cytokine production and proliferation by
spleen mononuclear cells from etoposide-treated mice*

| | IL-6 (pg/ml) | | TNF-α (pg/ml) | | Counts per minute (CPM) | |
|---|---|---|---|---|---|---|
| | Con A | TSST-1 | Con A | TSST-1 | Con A | TSST-10 |
| Etoposide-treated | 74 ± 15^{a)} | 10 ± 3 | 6 ± 0.5^{a)} | 6 ± 0.4^{a)} | 2183 ± 214^{a)} | 1117 ± 26^{a)}156 ± 9^{a)} |
| Controls | 309 ± 61 | 18 ± 2 | 124 ± 7.8 | 24 ± 0.33 | 2973 ± 3 603 | 14626 ± 4440452 ± 9 |

*Data are expressed as the mean ± SEM from 4 mice. Mice were injected with 12.5 mg/kg of etoposide 4 days a week for 4 weeks. Cells were stimulated with toxic shock syndrome toxin 1 (TSST-1) and concanavalin A (Con A).
^{a)}p < 0.01 versus controls

TABLE 10
Levels of TNF (pg/ml) in supernatants from
a murine macrophage cell line (IC-21)*

| | LPS (ng/ml) | | | | | IFN-γ (u/ml) | | |
|---|---|---|---|---|---|---|---|---|
| Etoposide (μM) | 0 | 1 | 10 | 100 | 1000 | 10 | 50 | 100 |
| 0 | 79 | 263 | 3850 | 11300 | 14500 | 61 | 55 | 68 |
| 0.01 | 66 | 207 | 3050 | 11900 | 12375 | 52 | 56 | 65 |
| 0.1 | 24 | 72 | 1125 | 2325 | 3650 | 20 | 32 | 34 |

*Data are expressed as the mean of two experiments with similar results. IC-21 cells were treated with etoposide for 24 hours, followed by stimulation with LPS and IFN-γ ?for another 24 h.

TABLE 11
Levels of MIP-1α (pg/ml) in supernatants
from a murine macrophage cell line (IC-21)*

| | IFN-γ(u/ml) | | | |
|---|---|---|---|---|
| Etoposide (μM) | 0 | 10 | 50 | 100 |
| 0 | 7950 | 4175 | 1400 | 700 |
| 0.01 | 5700 | 4200 | 1075 | 800 |
| 0.1 | 3400 | 1950 | 900 | 488 |

*Data are expressed as the mean of two experiments with similar results. IC-21 cells were treated with etoposide for 24 hours, followed by stimulation with IFN-γ ?for another 24 h.

### FIGURE LEGENDS

FIG. 1. Severity of arthritis (A), and survival rates (B) of NMRI mice after intravenous injection of 1-2×10⁷ CFU of S. aureus LS-1. Mice were pretreated with etoposide (n=⁴⁵) or PBS (controls; n=46) starting three days prior to bacterial inoculation. *p<0.05˜**p<0.01; N.S., not significant.

FIG. 2. Proliferative responses of mouse spleen mononuclear cells incubated with different concentrations of etoposide and stimulated with Con A and TSST-1, respectively. Results represent means±SEM of four individually analysed spleens per group. *p4.0 I

FIG. 3. Proliferative responses of mouse spleen mononuclear cells incubated with Con A and TSST-1, respectively. The mice were treated once a day for three days with etoposide or PBS (n—per group); NS, not significant.

FIG. 4. Total numbers of circulating monocytes in mice treated with etoposide starting three days prior to i. v. inoculation with 1-2×10⁷ CFU of S. aureus (n=8-15 per group). P<0.001.

FIG. 5. Effect of etoposide treatment on arthritis frequency in CII-immunized mice. Mice were treated with etoposide two days prior to immunization and then 4 times per week (“Etoposide-pretreated”; n=19), or 40 days after immunization and then 4 times per week (“Etoposide-treated”; n—17), or left untreated (“Controls”; n=17).

FIG. 6. Levels of IL-6 in supernatants from macrophage cell line (IC-21). IC-21 cells were treated with etoposide at different concentration (0, 0.01, 0.1 ?M) for 24 h, followed by stimulation with LPS (FIG. 6A) or IFN-γ (FIG. 6B) for another 24 h. Pooled data from two experiments are provided.

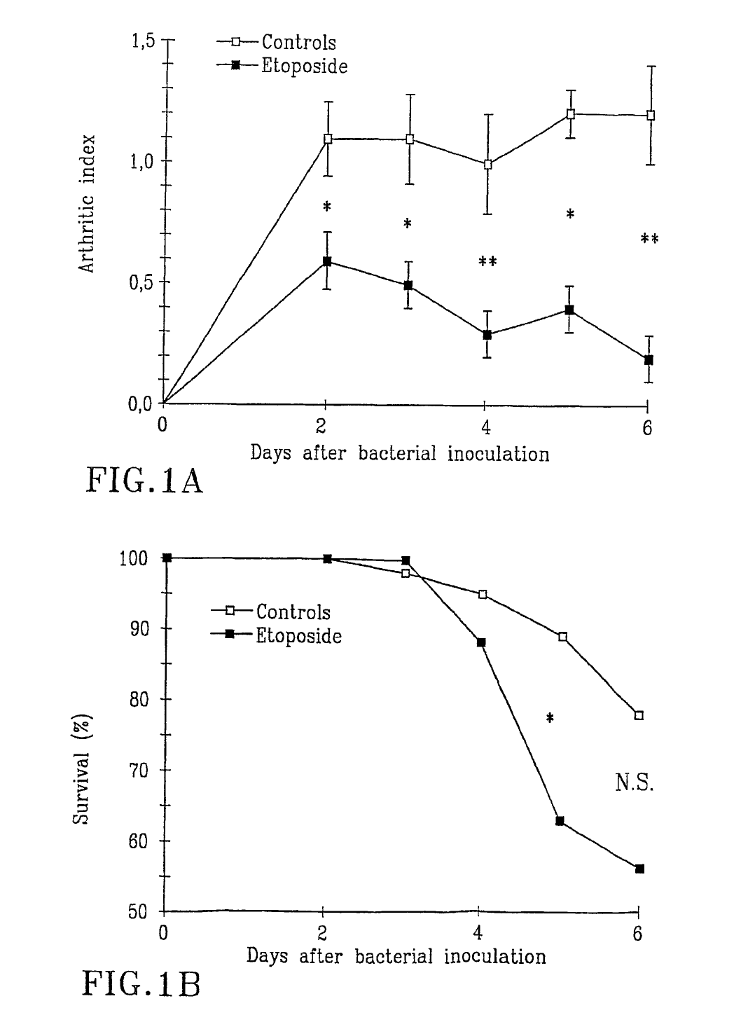

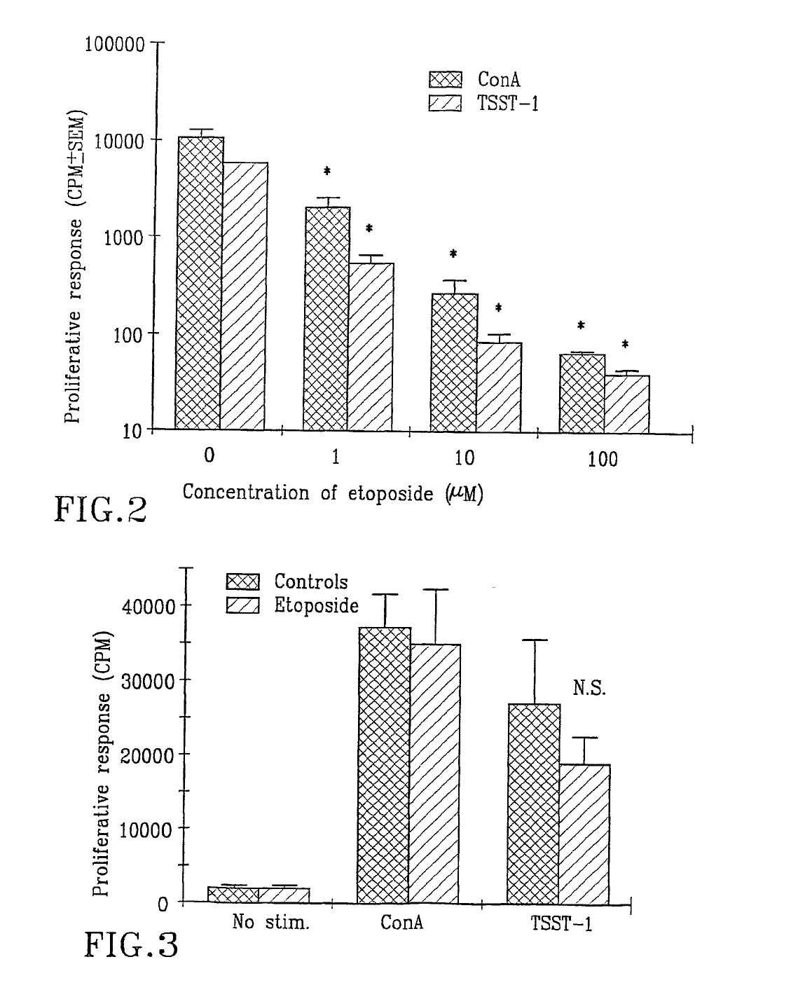

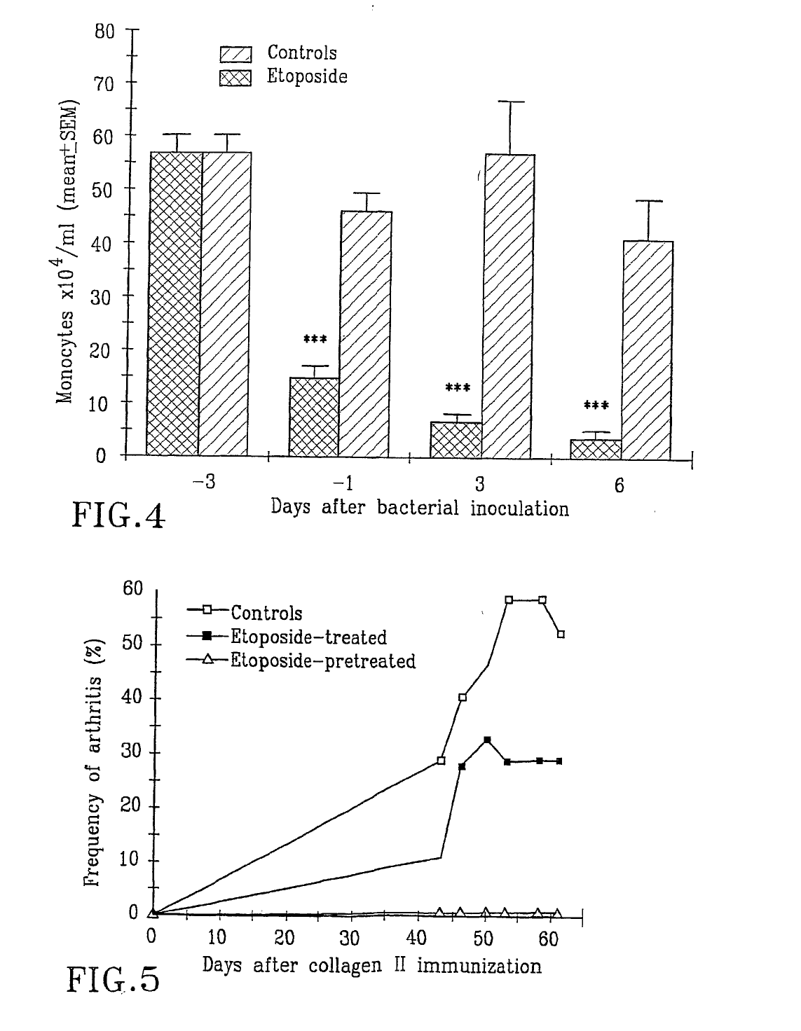

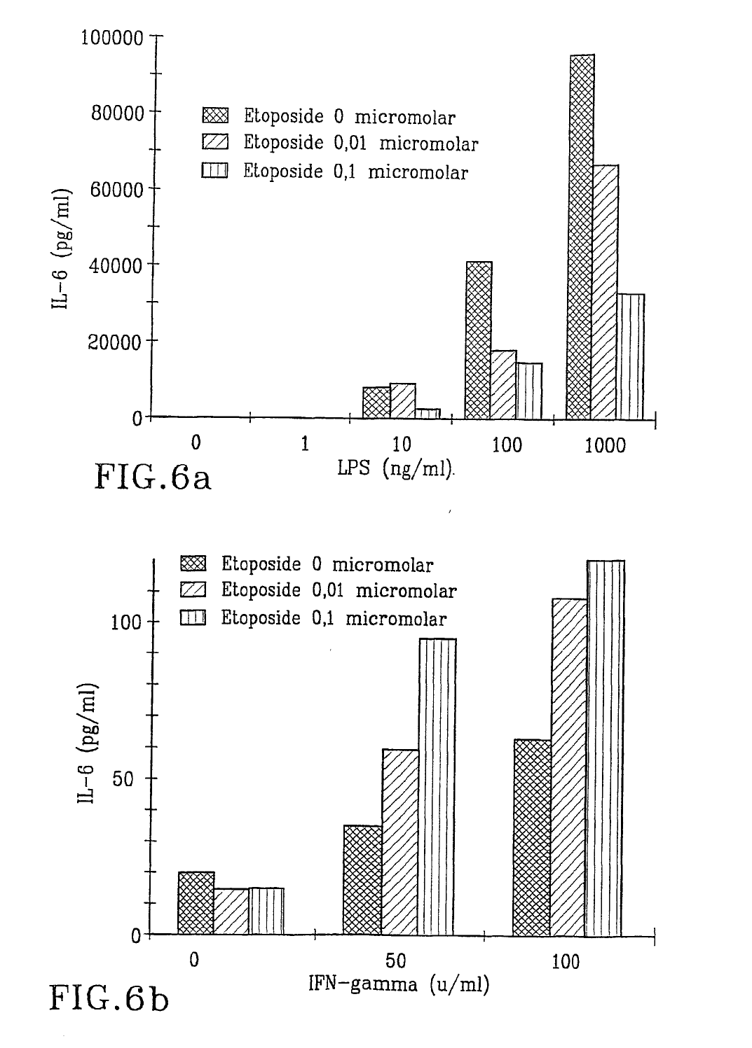

1. The use of etoposide at the manufacture of pharmaceutical compositions intended for the treatment of inflammations in mammals, including humans.

2. The use of etoposide according to claim 1 at the manufacture of pharmaceutical compositions intended for the treatment of arthritis in mammals, including humans.

3. The use according to claim 2, wherein the arthritis is rheumatoid arthritis.

4. The use according to claim 2, wherein the arthritis is septic arthritis.

5. The use of etoposide at the manufacture of pharmaceutical compositions intended for the treatment of inflammations in mammals, including humans, wherein the inflammation is selected from the group consisting of ulcerous colitis, asthmatic conditions, connecting tissue inflammations.

6. The use according to claim 1-5, wherein the pharmaceutical compositions are manufactured to provide a daily dose of etoposide of 0.2 to 0.7 mg per kg bodyweight (=12.5 to 50 mg per m² body area).

7. The use according to claims 1-6, wherein the pharmaceutical composition comprises 1-99% by weight of etoposide.

8. The use according to claims 1-7, wherein the etoposide is present in a combination with an antibiotic agent.

9. Method for treating inflammations, such as arthritis, whereby a therapeutically active amount of etoposide is administered to mammals, including humans, suffering from symptoms and signs of arthritis.

10. Method according to claim 9, wherein etoposide is administered in a combination with an antibiotic agent for the treatment of septic arthritis.
